(19) United States
(12) Patent Application Publication
(10) Pub. No.: US 2021/0290624 A1
(43) Pub. Date: Sep. 23, 2021

(54) METHODS OF TREATING CANCERS CHARACTERIZED BY A HIGH EXPRESSION LEVEL OF SPINDLE AND KINETOCHORE ASSOCIATED COMPLEX SUBUNIT 3 (SKA3) GENE

(71) Applicant: UNIVERSITY HEALTH NETWORK, Toronto (CA)

(72) Inventors: Benjamin Haibe-Kains, Toronto (CA); David Cescon, Toronto (CA)

(21) Appl. No.: 17/056,275

(22) PCT Filed: May 22, 2019
(86) PCT No.: PCT/CA2019/050699

§ 371 Date: Nov. 17, 2020

Related U.S. Application Data
(60) Provisional application No. 62/675,228, filed on May 23, 2018.

(51) Int. Cl. A61K 31/519; A61K 31/437; A61K 31/44; A61K 31/517; C12Q 1/6886; A61P 35/00

(52) U.S. Cl. CPC A61K 31/519; A61K 31/437; A61K 31/44; C12Q 2600/106; C12Q 1/6886; A61P 35/00; C12Q 2600/158; A61K 31/517

(57) ABSTRACT

Provided herein are methods of treating cancers characterized by a high expression of SKA3 gene, such as: breast cancer, prostate cancer, endometrial cancer, ovarian cancer, brain cancer, skin cancer, thyroid cancer, lung cancer, mesothelioma cancer, bladder cancer, colorectal cancer, liver cancer, melanoma, glioblastoma, leukemia or lymphoma, comprising administering a therapeutically effective amount of a TTK inhibitor, such as: CFI-402257, BAY 1161909, BAY 1217389, AZ-3146, NMS-P715, TC Mps1 12, reversine, Mps1-IN-1, Mps1-IN-2, Mps1-IN-3, MPS BAY1, MPS BAY2a, MPS BAY2b, MPI-0479605, SP600125, S81694/NMS-P153; BOS172722; NTRC 0060-0; NTRC 1501-0; and a pharmaceutically acceptable salt thereof.

### CROSS-REFERENCE TO RELATED APPLICATIONS

This application claims the benefit of U.S. Provisional Application No. 62/675,228, filed May 23, 2018. The entire teachings of the aforementioned application are incorporated herein by reference.

### BACKGROUND OF THE INVENTION

Tyrosine threonine kinase (TTK), also known as Monopolar spindle 1 (MPS1), is a key regulator of the spindle assembly checkpoint (SAC), which functions to maintain genomic integrity. TTK is a conserved multi-specific kinase that is capable of phosphorylating serine, threonine and tyrosine residues when expressed in E. coli (Mills et al., J. Biol. Chem. 199222(5): 16000-16006). TTK mRNA is not expressed in the majority of physiologically normal tissues in human (Id). TTK mRNA is expressed in some rapidly proliferating tissues, such as testis and thymus, as well as in some tumors. For example, TTK mRNA was not expressed in renal cell carcinoma, but expressed in 50% of breast cancer samples, in testicular tumors and ovarian cancer samples. See Id. TTK is expressed in some cancer cell lines and tumors relative to normal counterparts (Id.; see also WO 02/068444 A1). TTK has emerged as a promising therapeutic target in human cancers, including triple negative breast cancer (TNBC). Several TTK inhibitors (TTKi) are being evaluated in clinical trials, and an understanding of the mechanism-mediating TTKi sensitivity and resistance could inform the successful development of this class of agents.

The development of targeted cancer therapeutics has intensified interest in the identification of biomarkers that have the potential to predict the response of patients to particular targeted therapies, thereby enabling the physician to tailor therapeutic regimens specific to each patient. Disclosed herein are patient populations that are particularly suited for treatment with a TTK inhibitor.

### SUMMARY OF THE INVENTION

Based in part on the discovery that cancer cell lines expressing higher levels of SKA3 are more responsive to TTK inhibition (see FIG. 2-4), provided herein are methods of selecting patients which are likely to be responsive to treatment with TTK inhibition (i.e., those expressing higher levels of SKA3), and then treating said patients with one or more TTK inhibitors. Thus, in one aspect, the present disclosure provides a method of treating a patient with a cancer characterized by a high expression level of SKA3.

The present teachings provide methods for treating a patient with a cancer characterized by a high expression level of SKA3 gene, the method comprises administering to the patient a therapeutically effective amount of a TTK inhibitor.

The present teachings also provide methods of identifying a patient that is likely to be responsive to a TTK inhibitor. The method comprises providing a sample from a cancer patient; determining SKA3 gene expression level in the sample; and administering to the patient with a therapeutically effective amount of a TTK inhibitor if the patient's cancer exhibits a high expression level of SKA3.

### BRIEF DESCRIPTION OF THE DRAWINGS

FIG. 1 illustrates distribution of area above the drug dose-response curve (AAC) for CFI-402257 across the panel of 52 breast cancer cell lines.

FIG. 2 is a volcano plot representing the strength (estimate) and significance (−log₁₀(p-value)) of all the univariate association between genes and drug sensitivity (AAC).

FIG. 3 illustrates the association between SKA3 expression and compound CFI-402257 sensitivity (as measured by AAC) with respect to molecular subtypes of breast cancer cell lines.

FIG. 4 is a boxplot showing the distribution of drug sensitivity (Area Above the drug dose-response Curve [AAC]) for cell lines expressing SKA3 mRNA greater than the median expression (SKA3 high; blue box), and cell lines expressing SKA3 mRNA less or equal than the median expression (SKA3 low; red box). CI: Concordance Index; P-value: Statistical significance of the concordance index. It shows the significantly different drug sensitivity for cell lines expressing SKA3 less or greater than the median expression.

FIG. 5 are violin plots displaying the distribution and probability density of SKA3 expression levels across various TCGA tumor types and across breast tumor molecular subtypes. Only tumor types with 500 or more patients were included.

FIG. 6: is a plot showing SKA3 expression values (log 2(TPM+0.001; TPM was estimated using Kallisto) across 743 breast cancer patients (all subtypes except Luminal A as it is not represented in the panel of BC cell lines). The expression threshold was determined as the median (top 50%) of this large set of tumors. Black vertical arrow represents an example of tumor with high expression of SKA3.

### DETAILED DESCRIPTION OF THE INVENTION

The spindle and kinetochore-associated (SKA) protein complex is a heterotrimeric complex (SKA1, SKA2, SKA3) that accumulates on spindle microtubules and at kinetochores after nuclear envelope breakdown, becoming most enriched on kinetochores at metaphase. In vitro studies show that domains on Skal and possibly Ska3 bind to microtubules (Welburn et al., Dev Cell 2009 (16), 374-385; Jeyaprakash et al., Mol Cell 2012 (46), 274-286; Schmidt et al., Dev Cell 2012 (23), 968-980). The spindle and kinetochore-associated subunit 3 (SKA3) is a gene encoding a component of the spindle and kinetochore-associated protein complex that regulates microtubule attachment to the kinetochores during mitosis. The encoded protein localizes to the outer kinetochore and may be required for normal chromosome segregation and cell division.

In one aspect, the cancer is determined to exhibit a high expression level of SKA3 prior to treatment with a therapeutically effective amount of a TTK inhibitor. This determination can be made by routine diagnostic methods which obtain cancer cells from a patient. These methods include, but are not limited to, biopsy, blood tests, and other diagnostic methods which obtain samples of cancer cells such as tissue samples, circulating tumor cells or biomolecules characteristic of cancer such as circulating nucleic acids. The expression level of SKA3 in the cancer cells is then determined. Determining if the cancer exhibits a high expression level of SKA3 is by methodology known in the art, for example, by determining SKA3 expression levels in the isolated cancer cells by RNA sequencing (RNA-Seq), microarray, quantitative PCR, or NanoString™ gene expression panels, or SKA3 protein by immunohistochemistry, flow cytometry, immunocytochemistry or Western blot. See e.g., RT-qPCR analysis discussed below. In one embodiment, the methods disclosed herein further comprise a step of performing a biopsy of the patient's cancer prior to treatment and determining from the cancer cells isolated from the biopsy if the cancer (cancer cells) exhibits a high expression level of SKA3.

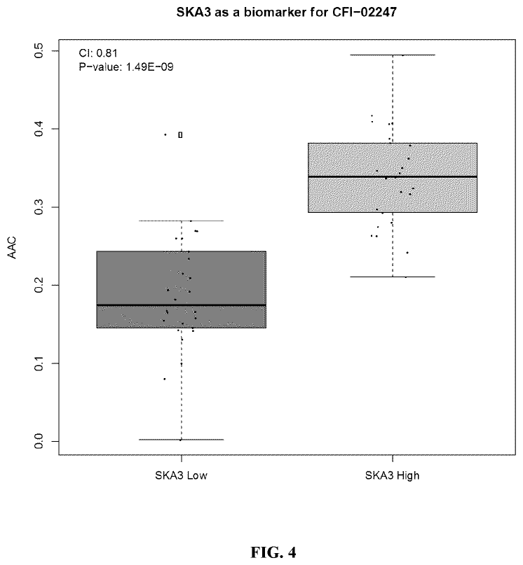

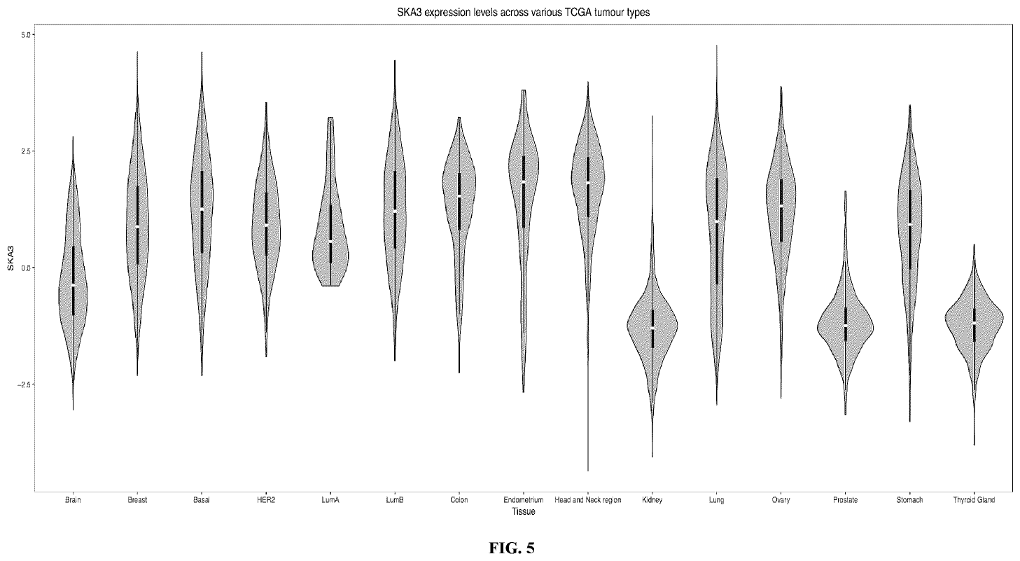

In another aspect, the invention is a method of treating a patient with a cancer comprising providing cancer cells from the cancer patient; determining the expression level of SKA3 in the cancer cells (see FIG. 4-5); and administering to the patient a therapeutically effective amount of a TTK inhibitor, if the patient's cancer (cancer cells) exhibits a high expression level of SKA3. In one embodiment, the method further comprising excluding the patient from administration of a TTK inhibitor if the patient's cancer (cancer cells) does not exhibit a high expression level of SKA3. The cancer cells used in the present invention can be obtained from a sample which is, but not limited to a sample of tissue, blood (including blood fractions), lymphatic fluid, sputum, feces, urine, bronchial lavage, or other body fluid.

In another aspect, provided herein is a method of selecting a patient who is likely to respond to treatment with a TTK inhibitor, said method comprising determining the expression level of SKA3 of a cancer of the patient, wherein the patient is likely to respond to treatment if the expression level of SKA3 by the cancer is high.

In another aspect, provided herein is a method of treating a patient with a cancer, comprising determining the expression level of SKA3 of the cancer and administering a therapeutically effective amount of a TTK inhibitor if the expression level of SKA3 by the cancer is high, and treating the patient with an anti-cancer therapy other than a TTK inhibitor if the patient's cancer does not exhibit a high expression level of SKA3.

In one aspect, the high expression level of SKA3 is characterized by an expression level falling within the top 50% of SKA3 expression levels of the cancer cells from the same cancer type in a random population of patients. The SKA3 expression level can be obtained from methods suitable for determining SKA3 expression levels, such as, e.g., expression levels derived from RNA-sequencing such as normalized read counts and TPM (Transcripts Per Million) or normalized cycle threshold (Ct) levels from RT-PCR measurements] for SKA3 robustly standardized (quantiles 2.5% and 97.5% set to −1 and +1, respectively).

As used herein “high expression” means an expression level falling within the top 5%, 10%, 15%, 20%, 25%, 30%, 40%, 45%, or 50% of the expression levels. “Top 50%”, for example, can be obtained by collecting expression levels of SKA3 from the cancer cells (e.g., from tissue samples) of a random population of subjects, e.g., at least 25 subjects, at least 50 subjects, at least 100 subjects, at least 500 subjects, at least 1000 subjects or the like, having the same cancers and then assessing whether the expression level of a new subject falls within the top 50% percentile. The expression level can be, for example, the level of SKA3, which can be determined as described in Example 1 in the materials and methods section.

In an alternative, “high expression” refers to a level of SKA3 in the cancer from the patient above a defined reference level of 25%, 30%, 40%, 50%, 60%, 70%, 80%, 85%, 90%, 95%, 100%, 150%, 200%, 250% or greater, determined by the methods described herein, as compared to the reference level.

“Reference level” refers to an average SKA3 expression level determined in cells of the same cell type as the cancer obtained from a population of healthy individuals without the cancer. In an alternative aspect, the reference level can be determined in non-cancerous cells of the same cell type as the cancer obtained from the patient.

In one aspect, the reference level can be obtained by determining the average normalized SKA3 expression [which can be obtained from methods suitable for determining SKA3 expression levels, such as, e.g., expression levels derived from RNA-sequencing such as normalized read counts and TPM (Transcripts Per Million) or normalized cycle threshold (Ct) levels from RT-PCR measurements] for SKA3 robustly standardized (quantiles 2.5% and 97.5% set to −1 and +1, respectively).

In one aspect, anti-cancer therapies other than a TTK inhibitor include, but are not limited to, surgery, radiation therapy, immunotherapy, endocrine therapy, gene therapy and administration of an anti-cancer agent other than a TTK inhibitor. In another aspect, anti-cancer therapies other than a TTK inhibitor include, but are not limited to, surgery, radiation therapy, immunotherapy, endocrine therapy, gene therapy, and epigenetic therapy, including the administration of an agent other than a TTK inhibitor.

Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body. Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy. In addition, immunotherapy drugs referred to as immune checkpoint inhibitors are designed to unshackle the patient's own immune system cells from attacking tumor cells. Examples include the drugs nivolumab (Opdivo) and pembrolizumab (Keytruda), which are monoclonal antibodies recognizing the PD-1 antigen, approved for the treatment of advanced classical Hodgkin lymphoma; atezolizumab (Tecentriq), a fully humanized monoclonal antibody against the protein programmed cell death-ligand 1 (PD-L1), approved for bladder cancer treatment; and ipilimumab (Yervoy), which is a monoclonal antibody that activates the immune system by targeting the CTLA-4 protein.

Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemo-labeled), a radioactive particle (radio-labeled), or a toxin (immunotoxin). Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer. Examples of the conjugated monoclonal antibodies include, but are not limited to radiolabeled antibody Ibritumomab tiuxetan (Zevalin), a monoclonal antibody against the CD20 antigen which delivers radioactivity directly to cancerous B lymphocytes and is used to treat, for example, B cell non-Hodgkin lymphoma; radiolabeled antibody Tositumomab (Bexxar), another monoclonal antibody recognizing the CD20 antigen, which is used to treat, for example, certain types of non-Hodgkin lymphoma; and immunotoxin Gemtuzumab ozogamicin (Mylotarg), a monoclonal antibody to CD33 linked to the cytotoxic agent calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies.

An additional example of passive immunotherapy, involving gene therapy, would include CAR (Chimeric antigen receptor) T-cell therapy, which involves genetically modifying the patient's own T cells to target and enhance their cancer-fighting ability. FDA approved CAR-T therapies include axicabtagene ciloleucel (Yescarta), which targets the CD19 antigen and is approved for the treatment of diffuse large B-cell lymphoma; and tisagenlecleucel (Kymriah), used for the treatment of relapsed/refractory B-cell precursor acute lymphoblastic leukemia.

In one aspect, immunotherapies that can be used in the present teachings include adjuvant immunotherapies. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); and combinations thereof, such as, for example, combinations of, interleukins, for example, IL-2 with other cytokines, such as IFN-alpha.

An endocrine therapy is a treatment that adds, blocks or removes hormones. For example, chemotherapeutic agents that can block the production or activity of estrogen have been used for treating breast cancer. In addition, hormonal stimulation of the immune system has been used to treat specific cancers, such as renal cell carcinoma and melanoma. In one embodiment, the endocrine therapy comprises administration of natural hormones, synthetic hormones or other synthetic molecules that may block or increase the production or activity of the body's natural hormones. In another embodiment, the endocrine therapy includes removal of a gland that makes a certain hormone.

A gene therapy is the insertion of genes into a subject's cell and biological tissues to treat diseases, such as cancer. Exemplary gene therapy includes, but is not limited to, a germ line gene therapy and a somatic gene therapy, including the genetic modification of patient-derived immune T-cells referred to as CAR-T cell therapy.

In one aspect, cancer therapies other than a TTK inhibitor are other anti-cancer agents. An “anti-cancer agent” is a compound, which when administered in an effective amount to a subject with cancer, can achieve, partially or substantially, one or more of the following: arresting the growth, reducing the extent of a cancer (e.g., reducing size of a tumor), inhibiting the growth rate of a cancer, and ameliorating or improving a clinical symptom or indicator associated with a cancer (such as tissue or serum components), or increasing longevity of the subject.

The anti-cancer agent suitable for use in the methods described herein include anti-cancer agents that have been approved for the treatment of cancer. In one aspect, the anti-cancer agent includes, but is not limited to, a targeted antibody, an immune checkpoint inhibitor, an angiogenesis inhibitor, an epigenetic agent, an alkylating agent, an antimetabolite, a vinca alkaloid, a taxane, a podophyllotoxin, a topoisomerase inhibitor, a hormonal antineoplastic agent and other antineoplastic agents.

Examples of alkylating agents useful in the methods of the present teachings include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful in the methods of the present teachings include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of plant alkaloids and terpenoids or derivatives thereof include, but are not limited to, vinca alkaloids (e.g., vincristine, vinblastine, vinorelbine, vindesine), podophyllotoxin, and taxanes (e.g., paclitaxel, docetaxel). Examples of a topoisomerase inhibitor includes, but is not limited to, irinotecan, topotecan, amsacrine, etoposide, etoposide phosphate and teniposide. Examples of antineoplastic agents include, but are not limited to, actinomycin, anthracyclines (e.g., doxorubicin, daunorubicin, valrubicin, idarubicin, epirubicin), bleomycin, plicamycin and mitomycin.

In one aspect, the anti-cancer agents that can be used in the present teachings include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents/drugs that can be used in the present teachings include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In one aspect, cancer therapies are anti-cancer agents suitable for treating leukemias. Exemplary treatments include, but are not limited to, Abitrexate® (Methotrexate), Arranon® (Nelarabine), Asparaginase Erwinia chrysanthemi, Blinatumomab, Blincyto® (Blinatumomab), Cerubidine® (Daunorubicin Hydrochloride), Clafen® (Cyclophosphamide), Clofarabine®, Clofarex® (Clofarabine), Clolar® (Clofarabine), Cyclophosphamide, Cytarabine, Cytosar-U® (Cytarabine), Cytoxan® (Cyclophosphamide), Dasatinib, Daunorubicin Hydrochloride, Doxorubicin Hydrochloride, Erwinaze® (Asparaginase Erwinia chrysanthemi), Folex® (Methotrexate), Folex PFS® (Methotrexate), Gleevec® (Imatinib Mesylate), Iclusig® (Ponatinib Hydrochloride), Imatinib Mesylate, Marclibo® (Vincristine Sulfate Liposome), Mercaptopurine, Methotrexate, Methotrexate LPF® (Methorexate), Mexate® (Methotrexate), Mexate-AQ® (Methotrexate), Nelarabine, Neosar® (Cyclophosphamide), Oncaspar® (Pegaspargase), Pegaspargase, Ponatinib Hydrochloride, Prednisone, Purinethol® (Mercaptopurine), Purixan® (Mercaptopurine), Rubidomycin® (Daunorubicin Hydrochloride), Spryce® 1 (Dasatinib), Tarabine PFS® (Cytarabine), Vincasar PFS® (Vincristine Sulfate), Vincristine Sulfate, Vincristine Sulfate Liposome, Hyper-CVAD, Arsenic Trioxide, Idamycin (Idarubicin Hydrochloride), Idarubicin Hydrochloride, Mitoxantrone Hydrochloride, Tabloid (Thioguanine), Thioguanine, Trisenox® (Arsenic Trioxide), Alemtuzumab, Ambochlorin® (Chlorambucil), Arzerra® (Ofatumumab), Bendamustine Hydrochloride, Campath® (Alemtuzumab), Chlorambucil, Fludara® (Fludarabine Phosphate), Fludarabine Phosphate, Gazyva® (Obinutuzumab), Ibrutinib, Idelalisib, Imbruvica® (Ibrutinib), Leukeran® (Chlorambucil), Linfolizin® (Chlorambucil), Mechlorethamine Hydrochloride, Mustargen® (Mechlorethamine Hydrochloride), Obinutuzumab, Ofatumumab, Rituxan® (Rituximab), Rituximab, Treanda® (Bendamustine Hydrochloride), Venclexta® (Venetoclax), Venetoclax, Zydelig® (Idelalisib), chlorambucil-prednisone, CVP, Bosulif (Bosutinib), Bosutinib, Busulfan, Busulfex (Busulfan), Hydrea® (Hydroxyurea), Hydroxyurea, Mechlorethamine Hydrochloride, Myleran® (Busulfan), Neosar (Cyclophosphamide), Nilotinib, Omacetaxine Mepesuccinate, Synribo® (Omacetaxine Mepesuccinate), and Tasigna® (Nilotinib).

TTK or MPS1 inhibitors described herein include e.g., small molecules that are capable of inhibiting tyrosine threonine kinase or monopolar spindle 1 activity. Inhibition can be measured in vitro, in vivo, or from a combination thereof. In one aspect, the TTK or MPS1 inhibitors in the methods described herein include, but are not limited to, those described in WO2014075168, WO2015070349, WO2013053051, WO2014056083, WO 2009024824, WO 2013087579, WO 2014198647, WO 2014195408, WO 2014009219, WO 2014131739, WO 2016034507, WO 2009156315, WO 2010007756, Hewitt et al. J Cell Biol (2010) 190: 25-34, Wengner et al. Mol Cancer Ther (2016) 15: 583-592, Tardif et al. Mol Cancer Ther (2011) 10: 2267-2275, Jemaa et al. Cell Death Differ (2013) 20: 1532-1545, Kwiatkowski et al. Nat Chem Biol (2010) 6: 359-368, Tannous et al. J Natl Cancer Inst (2013) 105: 1322-1331, Colombo et al. Cancer Res (2010) 70: 10255-10264, Maia et al. Ann Oncol (2015) 26: 2180-2192, Santaguida et al. J Cell Biol (2010) 190: 73-87, and Schmidt et al. EMBO Rep (2005) 6: 866-872.

In one aspect, the TTK/MPS1 inhibitors in the methods described herein are

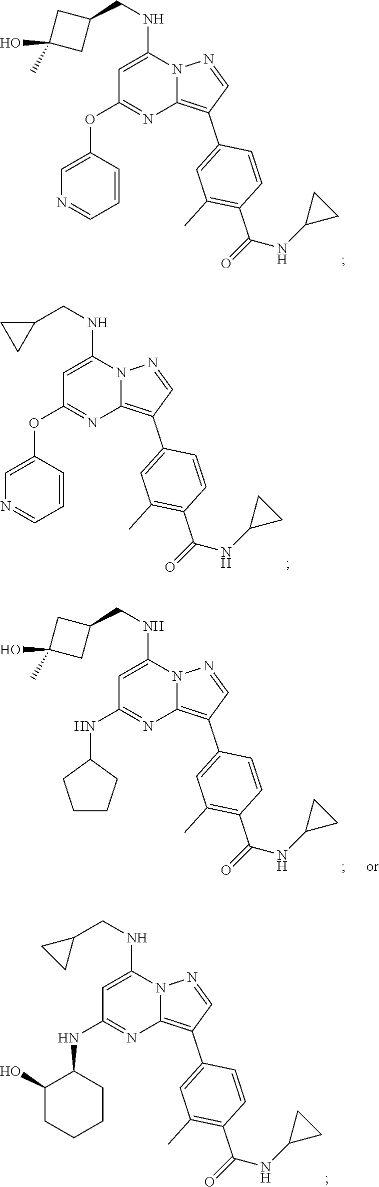 [SMILES: Cc1cc(-c2cnn3c(NC[C@H]4C[C@@](C)(O)C4)cc(Oc4cccnc4)nc23)ccc1C(=O)NC1CC1 ; Cc1cc(-c2cnn3c(NCC4CC4)cc(Oc4cccnc4)nc23)ccc1C(=O)NC1CC1 ; Cc1cc(-c2cnn3c(NC[C@H]4C[C@@](C)(O)C4)cc(NC4CCCC4)nc23)ccc1C(=O)NC1CC1 ; Cc1cc(-c2cnn3c(NCC4CC4)cc(N[C@H]4CCCC[C@H]4O)nc23)ccc1C(=O)NC1CC1]

or a pharmaceutically acceptable salt thereof.

In one aspect, the TTK/MPS1 inhibitors in the methods described herein are selected from the group consisting of

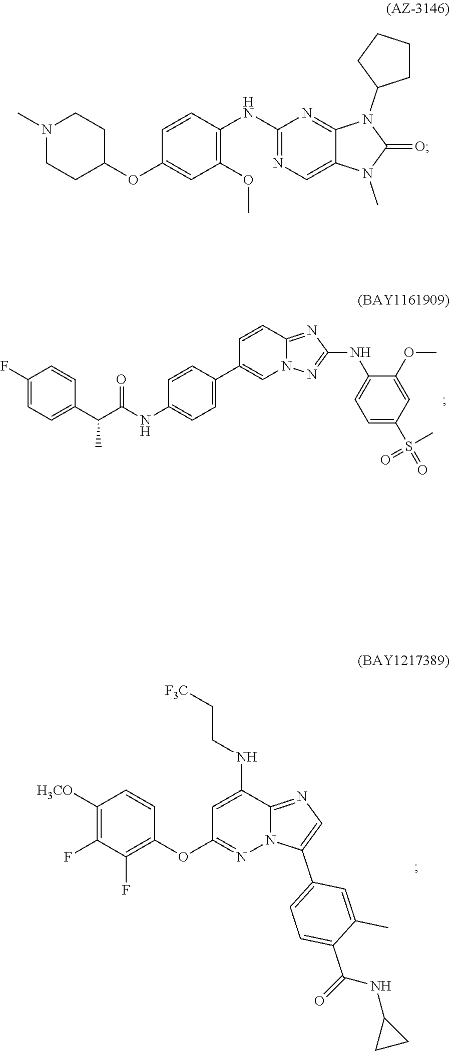 [SMILES: COc1cc(OC2CCN(C)CC2)ccc1Nc1ncc2c(n1)n(C1CCCC1)c(=O)n2C ; COc1cc(S(C)(=O)=O)ccc1Nc1nc2ccc(-c3ccc(NC(=O)[C@H](C)c4ccc(F)cc4)cc3)cn2n1 ; ";" ; COc1ccc(Oc2cc(NCCC(F)(F)F)c3ncc(-c4ccc(C(=O)NC5CC5)c(C)c4)n3n2)c(F)c1F ; ";"]

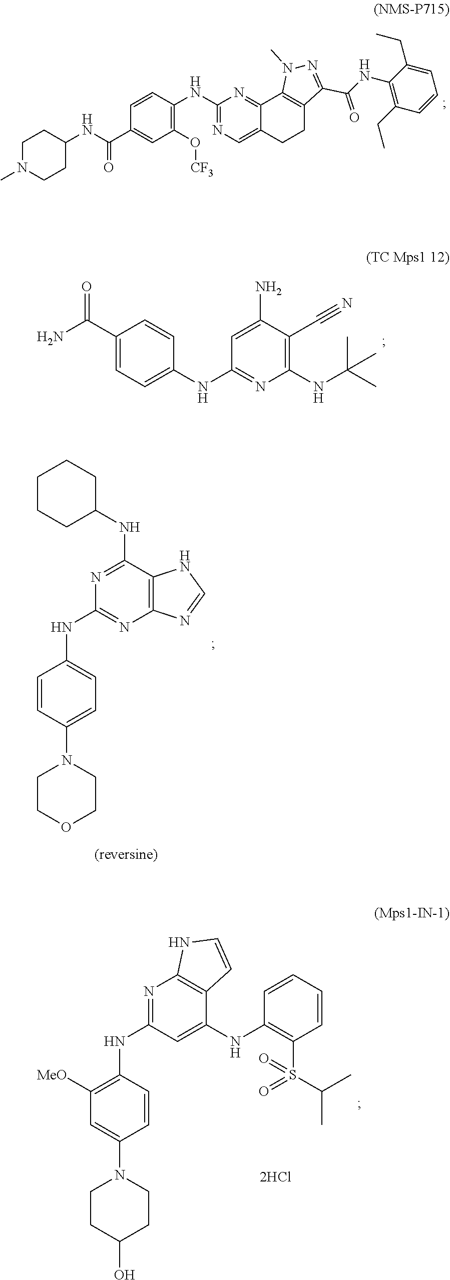

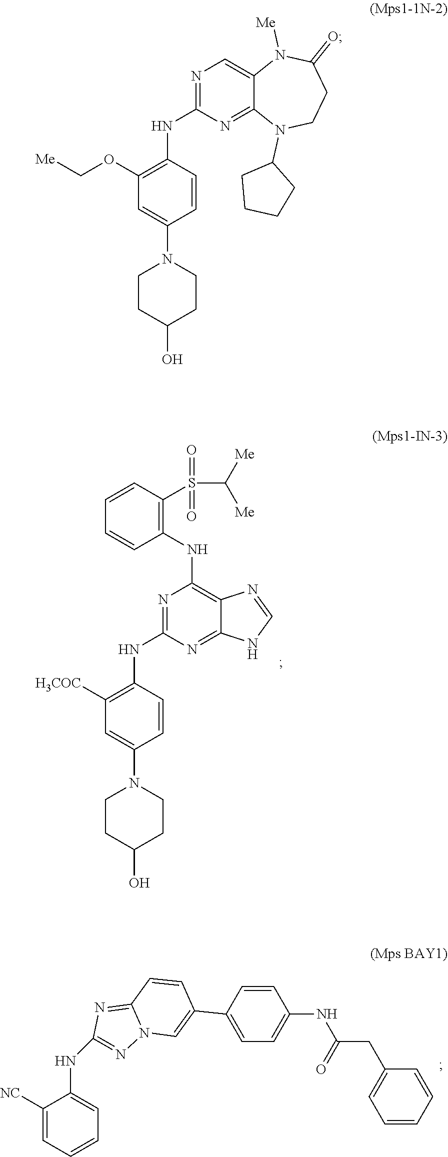

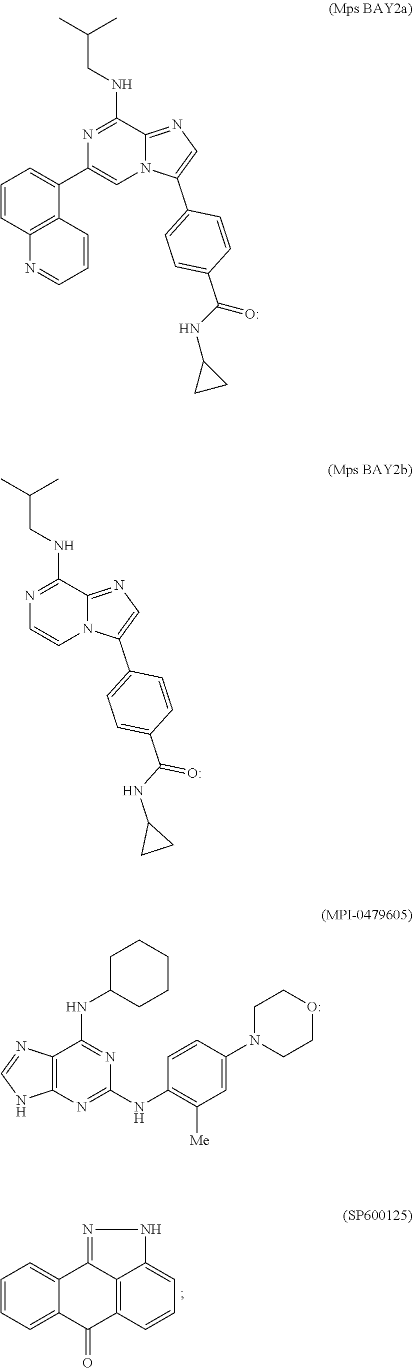

S81694/NMS-P153; BOS172722; NTRC 0060-0; NTRC 1501-0; or a pharmaceutically acceptable salt thereof.

When the stereochemistry about the cyclobutyl or cyclohexyl in the TTK/MPS1 inhibitors described herein is indicated by structure only, the structure is meant to depict the relative stereochemistry at one of the chiral centers in the cyclobutyl/cyclohexyl relative to the stereochemistry at the other chiral center, and not the absolute stereochemistry at either chiral center in the cyclobutyl or cyclohexyl. For example, when the stereochemistry about the cyclobutyl is depicted by structure only as being trans, the stereochemical purity of the compound with respect to the depicted trans configuration about the cyclobutyl is at least 85%, at least 90%, at least 95%, at least 97%, at least 98% or at least 99% by weight, i.e., the percent by weight of the TTK/MPS1 inhibitor in a composition having the trans stereochemistry at the cyclobutyl is at least 85%, at least 90%, at least 95%, at least 97%, at least 98% or at least 99% by weight. For example, the TTK/MPS1 inhibitor represented by the formula:

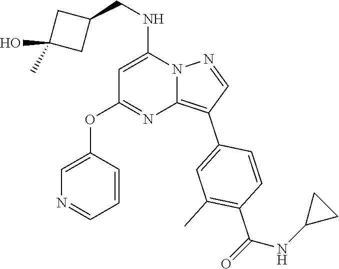 [SMILES: Cc1cc(-c2cnn3c(NC[C@H]4C[C@@](C)(O)C4)cc(Oc4cccnc4)nc23)ccc1C(=O)NC1CC1]

means that at least 85%, at least 90%, at least 95%, at least 97%, at least 98% or at least 99% by weight of the TTK/MPS1 inhibitor in a composition has the depicted trans configuration about the cyclobutyl; at least at least 85%, at least 90%, at least 95%, at least 97%, at least 98% or at least 99% by weight of the TTK/MPS1 inhibitor in the composition contains the other trans configuration as:

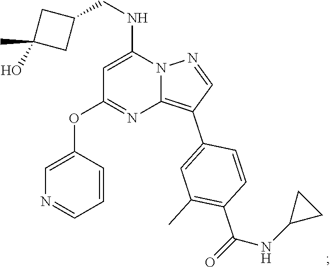 [SMILES: Cc1cc(-c2cnn3c(NC[C@H]4C[C@@](C)(O)C4)cc(Oc4cccnc4)nc23)ccc1C(=O)NC1CC1]

or at least 85%, at least 90%, at least 95%, at least 97%, at least 98% or at least 99% by weight of the compound in a composition is a mixture of the two trans configurations.

When the absolute stereochemistry of chiral centers in a TTK/MPS1 inhibitor of the methods described herein are indicated structurally and by “R” or “S” designations, it is to be understood that the depiction means the depicted stereoisomer at a stereochemical purity of at least 85%, at least 90%, at least 95%, at least 97%, at least 98% or at least 99% by weight, i.e., the percent by weight of the indicated stereoisomer of the TTK/MPS1 inhibitor represented in a composition. For example, the TTK/MPS1 inhibitor represented by the formula:

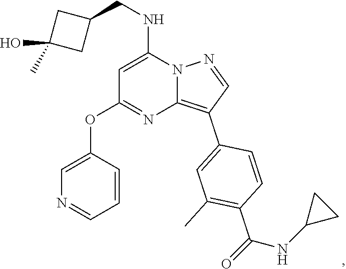 [SMILES: Cc1cc(-c2cnn3c(NC[C@H]4C[C@@](C)(O)C4)cc(Oc4cccnc4)nc23)ccc1C(=O)NC1CC1]

means at least 85%, at least 90%, at least 95%, at least 97%, at least 98% or at least 99% by weight of the TTK/MPS1 inhibitor in the composition contains of the depicted stereoisomer. When the structure being depicted by structure and by “R” or “S” designation is a single enantiomer, the enantiomeric purity is at least 95% (e.g., at least 96%, at least 97%, at least 98%, at least 99%, at least 99.5%, or at least 99.9%).

When a compound is depicted structurally without indicating the stereochemistry at a chiral center, it is to be understood that the structure includes either configuration at the chiral center or, alternatively, any mixture of configurations at the chiral center stereoisomers.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, or inhibiting the progress of a cancer, or one or more symptoms thereof, as described herein. Exemplary types of cancer include e.g., Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, neuroendocrine tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma, small cell neuroendocrine carcinomas and carcinoid tumors), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, diffuse intrinsic pontine glioma (DIPG), congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), fallopian tubes (carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma)), Hematologic: myeloid leukemia (acute and chronic), acute lymphoblastic leukemia, chronic lymphocytic leukemia, myeloproliferative diseases (primary myelofibrosis, polycythemia vera, essential thrombocythemia), multiple myeloma, myelodysplastic syndrome, Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, Merkel cell carcinoma, lipoma, angioma, dermatofibroma, keloids, psoriasis; and Adrenal glands: neuroblastoma.

In one aspect, the cancer characterized by a high expression of SKA3 is selected from the group consisting of breast cancer, prostate cancer, endometrial cancer, ovarian cancer, brain cancer, skin cancer, thyroid cancer, lung cancer, mesothelioma cancer, bladder cancer, colorectal cancer, liver cancer, melanoma, glioblastoma, leukemia and lymphoma. In one embodiment, the cancer is breast cancer. In one embodiment, the cancer is triple negative breast cancer. In another embodiment, the cancer is luminal breast cancer. In another embodiment, the cancer is HER positive breast cancer. In another embodiment, the cancer is hepatocellular carcinoma, ovarian cancer, mesothelioma, or lung cancer.

In another aspect, the cancer characterized by a high expression of SKA3 is selected from angiosarcoma, fibrosarcoma, rhabdomyosarcoma, liposarcoma, myxoma, rhabdomyoma, fibroma, lipoma, teratoma, squamous cell carcinoma, undifferentiated small cell carcinoma, undifferentiated large cell carcinoma, adenocarcinoma, alveolar carcinoma, bronchial adenoma, sarcoma, lymphoma, chondromatous hamartoma, mesothelioma, leiomyosarcoma, carcinoma, ductal adenocarcinoma, insulinoma, glucagonoma, gastrinoma, carcinoid tumors, vipoma, carcinoid tumors, Kaposi's sarcoma, leiomyoma, Merkel cell carcinoma, hemangioma, lipoma, neurofibroma, fibroma, tubular adenoma, villous adenoma, hamartoma, Wilm's tumor, transitional cell carcinoma, seminoma, teratoma, embryonal carcinoma, teratocarcinoma, choriocarcinoma, interstitial cell carcinoma, fibroma, fibroadenoma, adenomatoid tumors, lipoma, hepatocellular carcinoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, reticulum cell sarcoma, multiple myeloma, malignant giant cell tumor chordoma, osteochronfroma, benign chondroma, chondroblastoma, chondromyxofibroma, osteoid osteoma, giant cell tumors, osteoma, hemangioma, granuloma, xanthoma, osteitis deformans, meningioma, meningiosarcoma, gliomatosis, astrocytoma, medulloblastoma, glioma, ependymoma, germinoma, glioblastoma multiform, oligodendroglioma, schwannoma, retinoblastoma, congenital tumors, spinal cord neurofibroma, meningioma, endometrial carcinoma, cervical carcinoma, pre-tumor cervical dysplasia, ovarian carcinoma, granulosa-thecal cell tumors, Sertoli-Leydig cell tumors, intraepithelial carcinoma, clear cell carcinoma, squamous cell carcinoma, acute myeloid leukemia, chronic myeloid leukemia, acute lymphoblastic leukemia, chronic lymphocytic leukemia, myeloproliferative diseases, myelodysplastic syndrome, Hodgkin's disease, non-Hodgkin's lymphoma (malignant lymphoma), and neuroblastoma.

The term “pharmaceutically acceptable carrier, adjuvant, or vehicle” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a TTK inhibitor described herein in the composition will also depend upon the particular compound in the composition.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### EXEMPLIFICATION

### Example 1

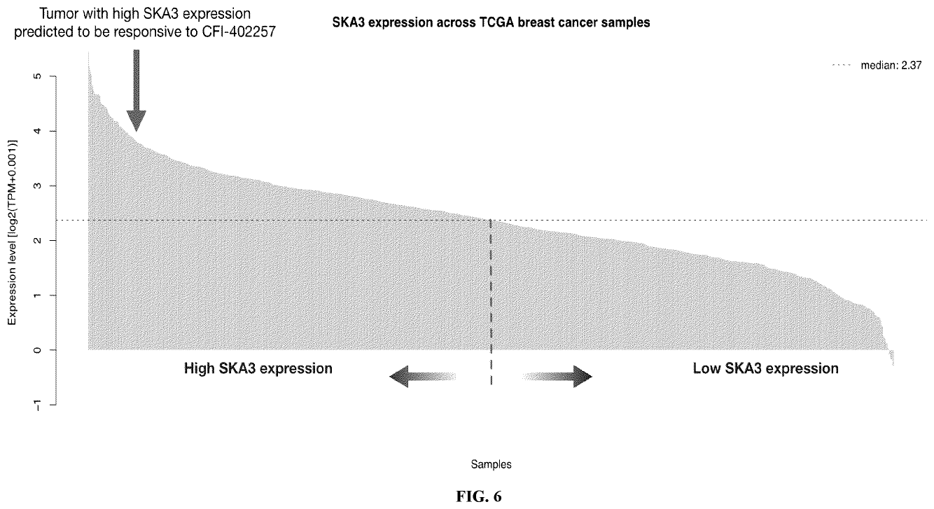

A breast cancer cell line panel, previously described (Marcotte et al., Cell, 164(1-2), 2016293-309), was assembled for in vitro drug sensitivity profiling, and mated with available gene expression data. The RNA-seq reads were aligned to the Ensembl Genome Reference Consortium release GRCh38 using Kallisto pipeline (Bray et al., Nature Biotechnology, 201634(5), 525-527). Expression values were computed as log₂(TPM+0.001), where TPM represents the number of transcripts per million mapped reads units which control for sequence length and sequencing depth. The status of SKA3 expression (high vs low expression) can be determined from a reference population such as the breast cancer TCGA cohort (see FIG. 6).

Response of breast cancer cell lines to compound CFI-402257 (a TTK inhibitor, see the structure below) was evaluated using sulforhodamine B (SRB) assays. The compound was synthesized as previously described (See Liu et al., ACS Medicinal Chemistry Letters, 20167(7), 671-675).

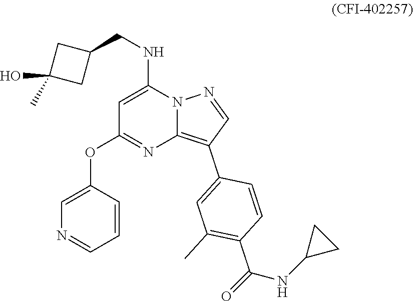 [SMILES: Cc1cc(-c2cnn3c(NC[C@H]4C[C@@](C)(O)C4)cc(Oc4cccnc4)nc23)ccc1C(=O)NC1CC1]

Cells were seeded in 96-well plates and treated with serial drug dilutions. After 5 days of treatment, cells were fixed, stained with SRB, solubilized, and absorbance quantified on a spectrophotometer. For all drug response assays, cell viabilities are reported as the proportion of growth in drug relative to DMSO treated conditions. The PharmacoGx pipeline was used to generate drug response metrics for each cell line including area above the drug dose-response curve (AAC), where higher AAC values represent higher drug sensitivity (Smirnov et al., Bioinformatics, 201632(8), 1244-1246).

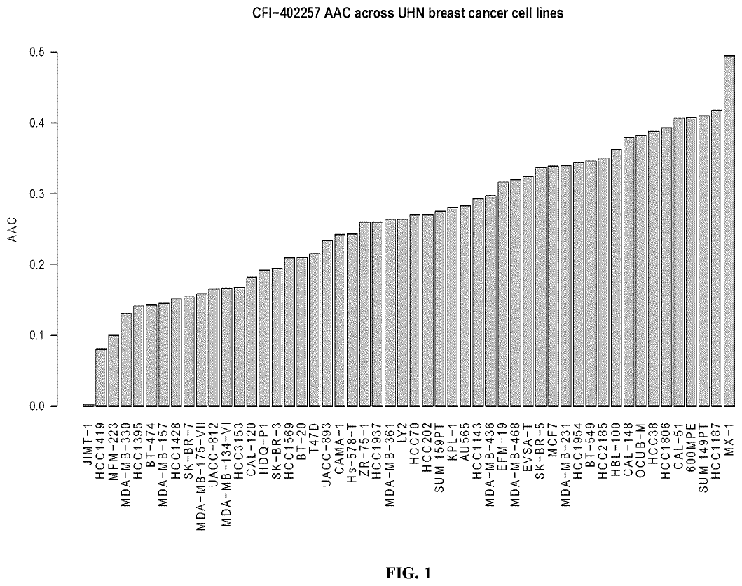

Differential in vitro sensitivity to compound CFI-402257 was observed across the breast cancer cell line panel (FIG. 1).

### Example 2

Cell Lines Expressing Higher Levels of SKA3 are Sensitive to TTK Inhibition

To identify gene expression robustly associated with drug sensitivity, a machine learning method was developed based on linear regression models. The model assumes a linear relationship between molecular features and drug responses. Although violation of this assumption may result in biased predictions, linear models are robust to variation or noise in the data, making them less prone to overfitting in a high-dimensional context such as pharmacogenomics. Therefore, the association between each molecular feature and response to a given drug is assessed by fitting linear models using the gene expression across cell lines as predictor variables, adjusted for tissue of origin of cancer cell lines, and their sensitivity values to the given drug as dependent variables. To assess the association of each gene to a given drug, two linear models were constructed for each dataset as following.

M₀:Y=β₀+β_{T}T (1)

M₁:Y=β₀+β_{T}T+β_{G}X_{G} (2)

Where T represents the tissues of origin as a vector of size N×1; Nis the number of cell lines; Y denotes the drug sensitivity vector of size N×1 containing the drug sensitivity values (AAC) of the cell lines treated by the drug of interest; X_{G} represents a vector of size N×1 of log₂ normalized TPM values for the expression of gene G across all the cell lines. The effect size of each association is quantified by β_{G}, which indicate the strength of associations between drug response and the molecular feature of interest, adjusted for tissue type. To estimate standardized coefficients from the linear model, the variables Y and X_{G} are scaled (standard deviation equals to one, mean equals to zero). The null model (Equation (1)) estimates the association between drug response and tissue source, as we previously showed that drug sensitivity in vitro is tissue specific (Yao et al., 2017). The model in Equations (2) estimates the strength and significance of the association between drug sensitivity and the gene-level expressions.

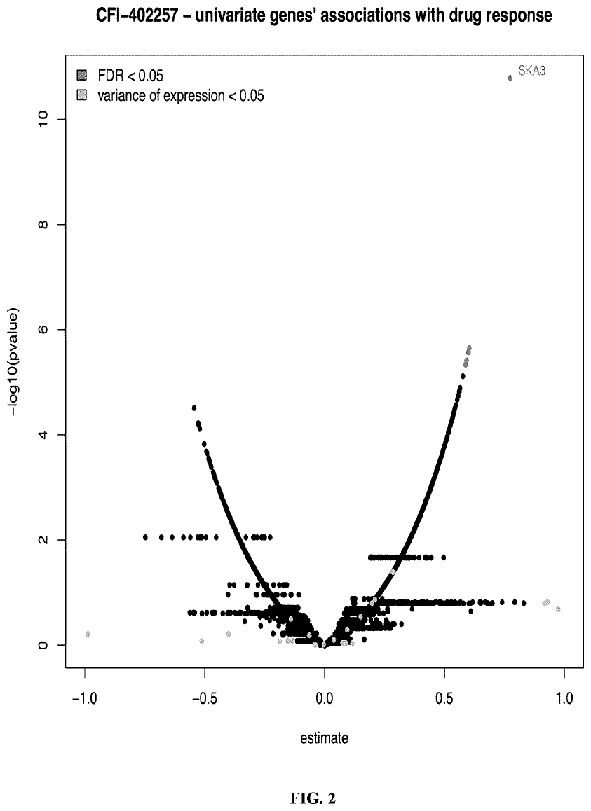

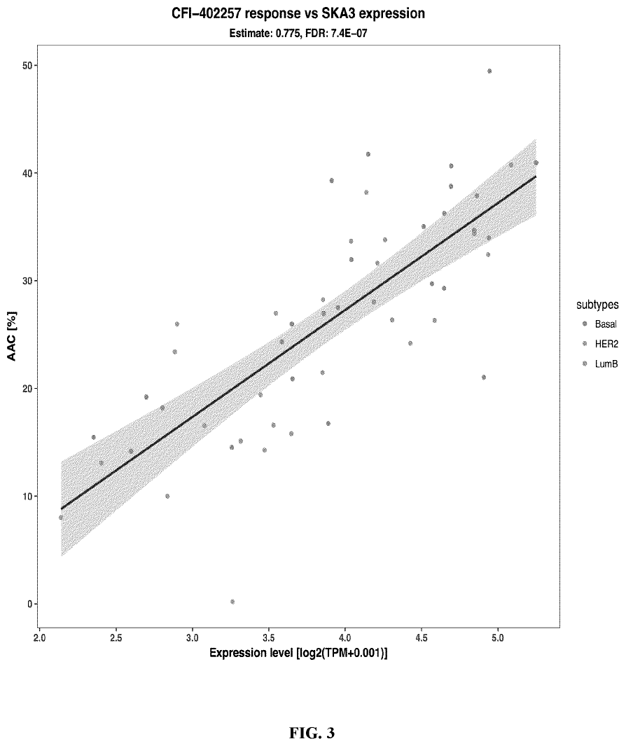

To identify univariate predictors of drug sensitivity, the association between AAC, a robust measure of drug response, and gene expression was performed. This analysis identified a strong and highly statistically significant association with the gene SKA3, substantially exceeding the association between drug sensitivity and any other single gene (FIG. 2). In order to evaluate the association between sensitivity to compound CFI-402257 and drug sensitivity within recognized molecular subtypes of breast cancers, the correlation between SKA3 expression and CFI-402257 sensitivity (as measured by AAC) was evaluated. A strong and statistically significant correlation was identified across all breast cancer cell lines, and within molecular subtypes represented in the cell line collection (FIG. 3).

Once the linear relationship between the continuous SKA3 expression and sensitivity (AAC) to CFI-402257 was established, we estimated the predictive of a simple binary classification of cell lines into SKA3 low/high based on median SKA3 expression across all the tested breast cancer cell lines (see FIG. 4). We estimated the predictive of such a binary classifier using the Concordance Index (CI=0.5 for random classifier and CI=1 denotes a perfect classification) [REF: Newson R: Confidence intervals for rank statistics: Somers' D and extensions. Stata Journal 6:309-334; 2006] and its significance (P-value) computed using the Noether formula [PMID: 22344892]. The SKA3 binary classification yielded a CI of 0.81 (P-value=1.5E-9), indicating that SKA3 binary expression is a strong biomarker of response to CFI-402257.

### Example 3

SKA3 Expression Varies in Different Cancer Cell Lines

In order to characterize the distribution of the SKA3 biomarker in clinical cancers, SKA3 gene expression from The Cancer Genome Atlas datasets was plotted in FIG. 5. Notably, only tumor types with 500 or more patients were included. FIG. 5 demonstrates significant variability in gene expression within breast cancers, as well as within other tumor types. Such variability in SKA3 expression in patient breast tumors suggest that individuals with high or low tumor SKA3 expression could be identified and would exhibit differential sensitivity to compound CFI-402257 or other TTK inhibitors.

These data suggest that SKA3 expression levels can be used as a criterion for selecting a subpopulation of cancer patients for treatment with a TTK inhibitor because of the broad range in values observed, with a distinct sub-population that can be characterized as “high” SKA3 expressors.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating a patient with cancer, wherein the cancer is characterized by a high expression level of Spindle and Kinetochore Associated Complex Subunit 3 (SKA3) gene, the method comprising administering to the patient a therapeutically effective amount of a TTK inhibitor.

2. The method of claim 1, wherein prior to treatment, the cancer was determined to have a high expression level of SKA3.

3. The method of claim 1, comprising the step of performing a biopsy of the patient's cancer prior to treatment and determining if the cancer exhibits a high expression level of SKA3 from cancer cells obtained from the biopsy.

4. A method of treating a patient with cancer, the method comprising:
(a) providing cancer cells from the cancer patient;
(b) determining SKA3 gene expression level in the cancer cells; and
(c) administering to the patient with a therapeutically effective amount of a TTK inhibitor if the patient's cancer exhibits a high expression level of SKA3.

5. The method of claim 4, further comprising excluding the patient from administration of a TTK inhibitor if the patient's cancer does not exhibit a high expression level of SKA3.

6. The method of claim 4, further comprising treating the patient with an anti-cancer therapy other than the TTK inhibitor if the patient's cancer does not exhibit a high expression level of SKA3.

7. The method of claim 1, wherein the high expression level of SKA3 is characterized by an expression level falling within the top 50% of SKA3 expression levels from a collection of cancer cells of the same cancer type in a random population of patients.

8. The method of claim 7, wherein the expression value falls within the top 25% of SKA3 expression levels from a collection of cancer cells from the same cancer type in a population of patients.

9. The method of claim 7, wherein the expression value falls within the top 10% of levels from a collection of cancer cells from the same cancer type in a random population of patients.

10. The method of claim 1, further comprising administering to the patient a therapeutically effective amount of a second anti-cancer agent.

11. The method of claim 1, wherein the cancer is is selected from the group consisting of breast cancer, prostate cancer, endometrial cancer, ovarian cancer, brain cancer, skin cancer, thyroid cancer, lung cancer, mesothelioma cancer, bladder cancer, colorectal cancer, liver cancer, melanoma, glioblastoma, leukemia and lymphoma.

12. The method of claim 1, wherein the cancer is breast cancer.

13. The method of claim 1, wherein the cancer is triple negative breast cancer.

14. The method of claim 1, wherein the cancer is luminal breast cancer.

15. The method of claim 1, wherein the cancer is HER positive breast cancer.

16. The method of claim 1, wherein the cancer is hepatocellular carcinoma, ovarian cancer, mesothelioma, or lung cancer.

17. The method of claim 1, wherein the TTK inhibitor is a compound represented by the following structural formula: 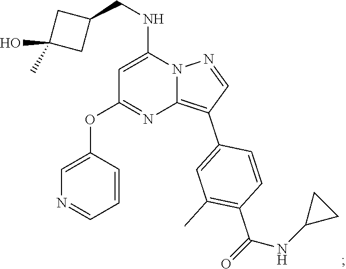 [SMILES: Cc1cc(-c2cnn3c(NC[C@H]4C[C@@](C)(O)C4)cc(Oc4cccnc4)nc23)ccc1C(=O)NC1CC1]
or a pharmaceutically acceptable salt thereof.

18. The method of claim 1, wherein the TTK inhibitor is a compound represented by the following structural formula: 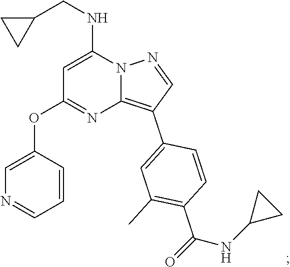 [SMILES: Cc1cc(-c2cnn3c(NCC4CC4)cc(Oc4cccnc4)nc23)ccc1C(=O)NC1CC1]
or a pharmaceutically acceptable salt thereof.

19. The method of claim 1, wherein the TTK inhibitor is a compound represented by the following structural formula: 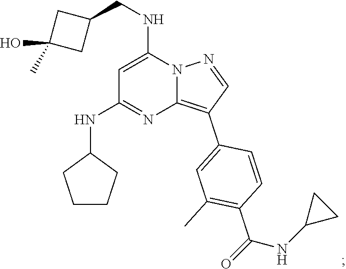 [SMILES: Cc1cc(-c2cnn3c(NC[C@H]4C[C@@](C)(O)C4)cc(NC4CCCC4)nc23)ccc1C(=O)NC1CC1]
or a pharmaceutically acceptable salt thereof.

20. The method of claim 1, wherein the TTK inhibitor is a compound represented by the following structural formula: 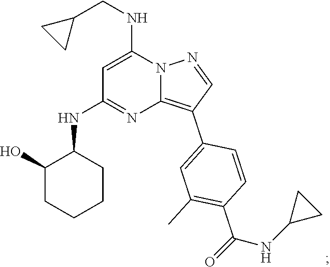 [SMILES: Cc1cc(-c2cnn3c(NCC4CC4)cc(N[C@H]4CCCC[C@H]4O)nc23)ccc1C(=O)NC1CC1]
or a pharmaceutically acceptable salt thereof.

21. The method of claim 1, wherein the TTK inhibitor is selected from the group consisting of 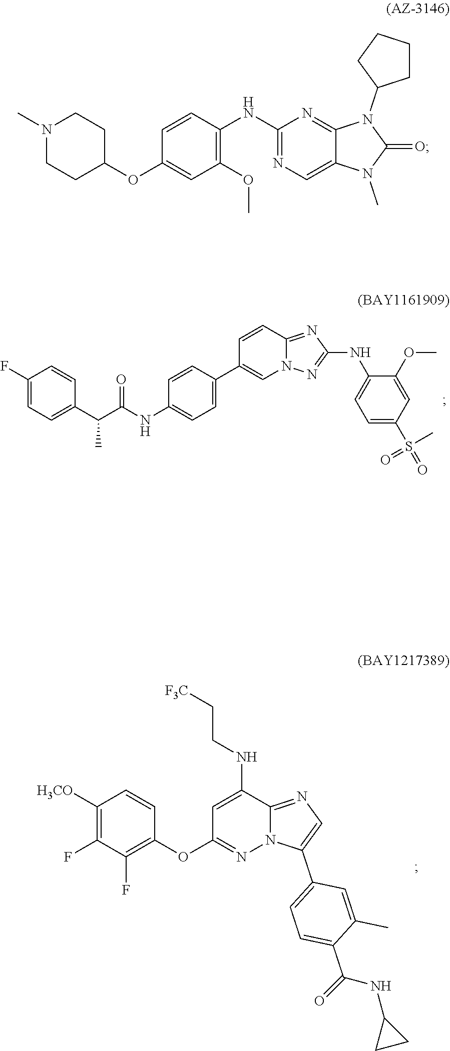 [SMILES: COc1cc(OC2CCN(C)CC2)ccc1Nc1ncc2c(n1)n(C1CCCC1)c(=O)n2C ; COc1cc(S(C)(=O)=O)ccc1Nc1nc2ccc(-c3ccc(NC(=O)[C@H](C)c4ccc(F)cc4)cc3)cn2n1 ; ";" ; COc1ccc(Oc2cc(NCCC(F)(F)F)c3ncc(-c4ccc(C(=O)NC5CC5)c(C)c4)n3n2)c(F)c1F ; ";"] 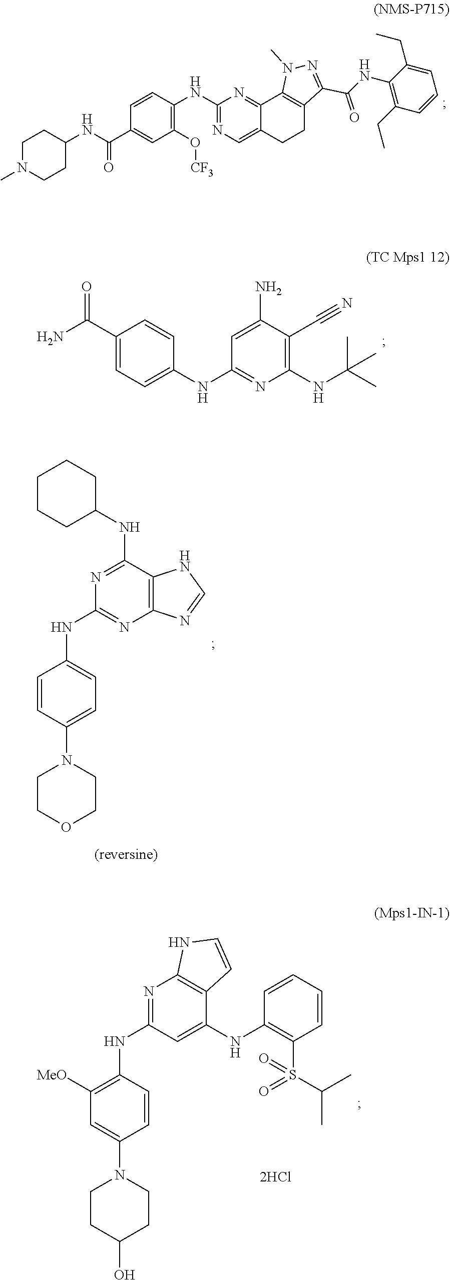 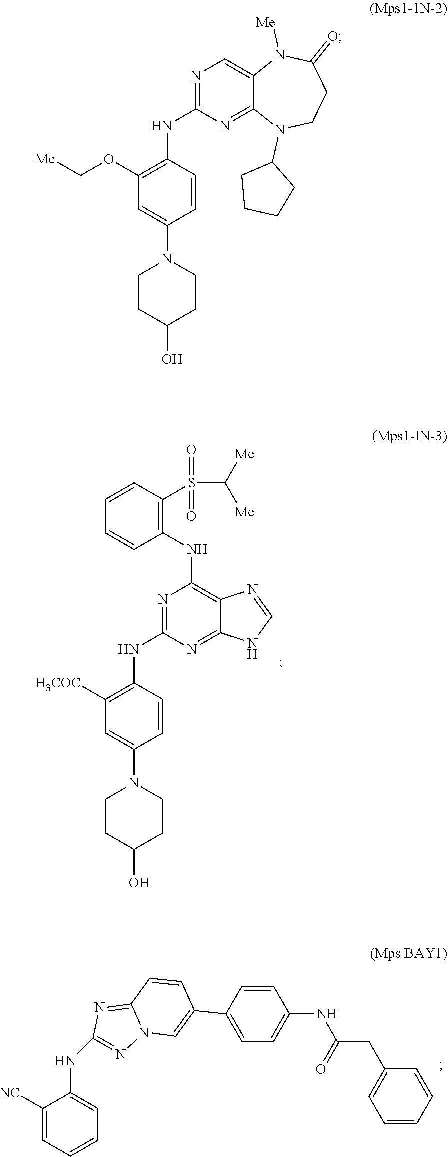 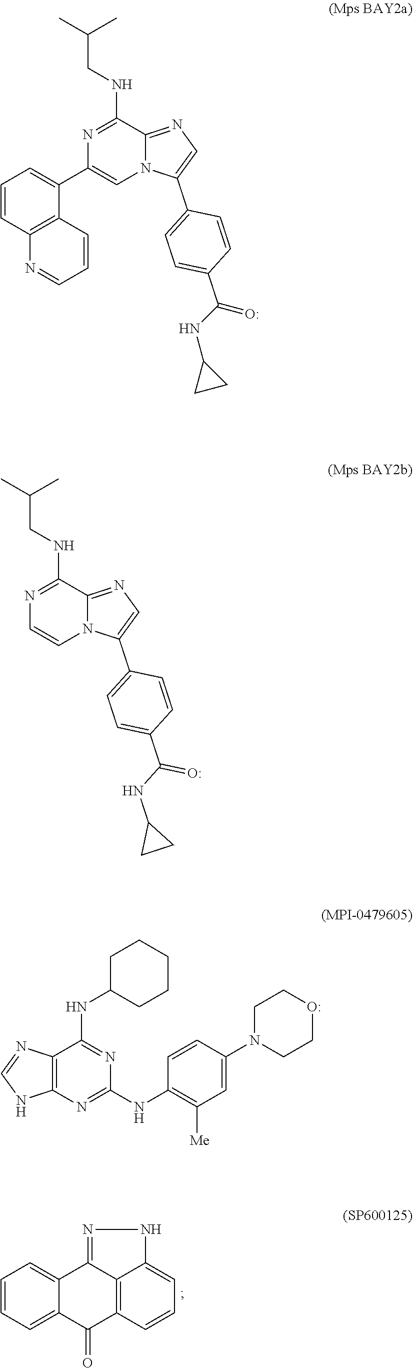
S81694/NMS-P153; BOS172722; NTRC 0060-0; NTRC 1501-0; and a pharmaceutically acceptable salt thereof.
